(19) United States
(12) Patent Application Publication
(10) Pub. No.: US 2009/0258590 A1
(43) Pub. Date: Oct. 15, 2009

(54) Hands-free smoke evacuation apparatus

(75) Inventor: Holly L. Prokash, Alpharetta, GA (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 12/082,431

(22) Filed: Apr. 11, 2008

(51) Int. Cl. A61B 1/00

(52) U.S. Cl. 454/49

(57) ABSTRACT

A hands-free apparatus for removing smoke from an operative site is provided. The apparatus includes a tube having one or more tube holders adapted to maintain the apparatus in communication with the operator's hand. During use, the apparatus rests on the operator's hand, without the need for the operator to manipulate the apparatus, and smoke or other atmospheric contaminants are transmitted through the tube into a suctioning device.

### BACKGROUND

Heating and/or burning of tissue during surgical procedures has become commonplace. An unwanted byproduct of such heating and/or burning, however, is surgical smoke. This surgical smoke may obscure the surgeon's field of vision. Additionally, the surgical smoke may generate odor and may otherwise be generally unpleasant and distracting to the entire surgical team, as well as the patient in those cases where the patient is awake during the surgical procedure. Moreover, the smoke plume may contain infectious agents that present a danger to persons in the operating room, and which can leave a lingering contamination within the operating area.

Smoke evacuation and filtering systems have been developed to remove smoke plumes from surgical sites. Such systems typically include a hose connected to a vacuum source or generator, i.e. a smoke evacuator or filtered wall suctioning device, and a suction wand connected to a hose that is placed at the site where the smoke is generated, or attached to an electrosurgical unit. Various filtration systems have been used in conjunction with such vacuum generators to remove odor and infectious agents. Although these smoke evacuation and filtration systems are generally effective, they present tactical problems because, the wand and hoses of known evacuation and filtration systems require the constant attention or activity of an attendant to hold the wand or the nozzle of the hose close to the surgical site. Additionally, attaching the wand and/or hoses to the electrosurgical unit may result in bulkiness of the surgical instrument(s) resulting in reduced dexterity of the surgeon or surgical assistant.

In this regard, during surgical procedures the surgical assistant or scrub nurse's hands are typically near the surgical site but his or her hands are also holding skin, tissue, retractors, or other instruments. This makes it difficult to consistently hold smoke evacuation tubing near the surgical site so that smoke can be effectively evacuated in order to minimized inhalation by the surgical staff and/or patient. In many cases, because of this difficulty, surgeons, scrub nurses, or surgical assistants simply do not use smoke evacuation tubing.

Thus, there remains a need for an effective surgical smoke removing apparatus which does not detract from the surgical assistant or scrub nurse's surgical duties, while still efficiently removing smoke from the surgical site.

### SUMMARY OF INVENTION

The present invention provides for a hands-free apparatus for removing smoke from an operative site. The apparatus includes a tube having a tube body. The tube body includes a distal end, a proximal end, a substantially cylindrical central portion, at least one distal opening, and a proximal opening. The apparatus further includes one or more holders attached to the tube body. The holder or holders are adapted to releasably engage with the covered or uncovered hand, wrist, forearm, or combinations thereof during use. The holder or holders may be made of a rubber material, an elastic material, a non-elastic material, a cohesive material, a pressure sensitive adhesive, one or more mechanical fasteners, Velcro, or a hook and loop fastener. Desirably, during use, the hand, wrist, and/or forearm may be covered with a surgical gown and/or glove. The apparatus further includes one or more attachment zones whereon the holders are attached to the tube body. During use, the distal opening of the tube is adapted to receive smoke therethrough and the proximal opening of the tube is adapted to be attached to a suctioning device.

Desirably, during use, the tube rests at least partially upon a dorsal portion of the hand, wrist, forearm, or combinations thereof. This allows the tube to be used by a nurse or surgical assistant without the tube interfering with the medical procedure. Additionally, the distal opening of the tube may define a screen. This screen allows for surgical smoke, particulate matter associated with surgical smoke, and air to be suctioned into the distal end of the tube, and helps to prevent surgical instruments and other materials associated with surgery from being suctioned into the tube.

Additionally, various portions of the tube may exhibit different properties than other portions. For example, the distal end of the tube may be made of corrugated material. When corrugated material is used, the distal end of the tube may be extended or retracted as necessary to facilitate non-interference with the surgical procedure. Additionally, the distal end and proximal end of the tube may exhibit a greater degree of rigidity than the substantially central portion. This allows the proximal portion to be securely connected into a suctioning device such as an evacuator or wall suctioning unit and, allows the distal portion to have the structural support necessary for use in close proximity to a surgical procedure. At the same time, the greater flexibility of the central portion allows the apparatus to ergonomically function with the movement of the hands, wrist, or forearm during a surgical procedure.

Further, it may be desirable for the distal opening to be constructed so that the tip of the distal opening may be closed when lateral force is exerted against a portion of the distal end that is proximal to the distal opening, similar to, for example, the closing of a milk carton. This will allow the suctioning mechanism to be shut off when it is not in use and further limit any chance of surgical materials beings accidentally suctioned into the tube. Additionally, it may be desirable for the distal end to be in the shape of a Y-connector. A Y-connector allows smoke, or other undesirable atmospheric substances to be advantageously suctioned into the tube by utilizing more than one opening. This could allow for quicker, more efficient suctioning.

Another aspect of the invention addresses a hands-free apparatus for removing smoke from an operative site. The apparatus includes a flexible tube having a tube body. The tube body includes a distal end, a proximal end, a substantially cylindrical central portion, at least one distal opening, and a proximal opening. The apparatus further includes a capture tube adapted to connect to the distal end of the flexible tube and adapted to received smoke therethrough. The apparatus also includes an evacuation tube adapted to connect to the proximal end of the flexible tube and adapted to evacuate smoke therethrough to a suctioning device adapted to capture smoke. Additionally, the apparatus includes one or more holders attached to the tube body. The holder or holders are adapted to releasably engage with the covered or uncovered hand, wrist, forearm, or combinations thereof during use. The holder or holders may be made of a rubber material, an elastic material, a non-elastic material, a cohesive material, a pressure sensitive adhesive, one or more mechanical fasteners, Velcro, or a hook and loop fastener. Desirably, during use, the hand, wrist, and/or forearm may be covered with a surgical gown and/or glove. The apparatus further includes one or more attachment zones whereon the holders are attached to the tube body.

Yet another aspect of the invention includes a method of using a hands-free apparatus for removing smoke from an operative site. The method includes providing a tube having a tube body comprising a distal end, a proximal end, a substantially cylindrical central portion, at least one distal opening, and a proximal opening. The method also includes attaching one or more holders to the tube body at one or more attachment zones so the tube body releasably engages with the hand, wrist, forearm, or combinations thereof during use. The method further includes receiving smoke through the distal opening of the tube and transmitting smoke through the proximal opening of the tube to a suctioning device.

### BRIEF DESCRIPTION OF DRAWINGS

FIG. 1 is a perspective view of a unibody hands free apparatus for removing smoke from an operative site.

FIG. 2 is a perspective view of a hands free apparatus for removing smoke from an operative site, the apparatus having separate capture and evacuation tubes.

FIG. 3 is a perspective view of a hands free apparatus for removing smoke from an operative site in use and located on the dorsal portion of the wearer's hand, wrist, and forearm.

FIG. 4A is a perspective view of a corrugated distal end of the hands free apparatus for removing smoke from an operative site.

FIG. 4B is a perspective view of a distal end of the hands free apparatus for removing smoke from an operative site, the distal portion being a Y-connector.

### DETAILED DESCRIPTION

The apparatus of the present invention provide for a hands-free apparatus for removing smoke from an operative site. This apparatus allows for a surgical assistant or nurse to effectively suction smoke away from the surgical site, while not interfering with the ability of the surgical assistant or nurse to participate in the surgical procedure.

The invention will be described with reference to the following description and figures which illustrate certain embodiments. It will be apparent to those skilled in the art that these embodiments do not represent the full scope of the invention which is broadly applicable in the form of variations and equivalents as may be embraced by the claims appended hereto. Furthermore, features described or illustrated as part of one embodiment may be used with another embodiment to yield still a further embodiment. It is intended that the scope of the claims extend to all such variations and embodiments.

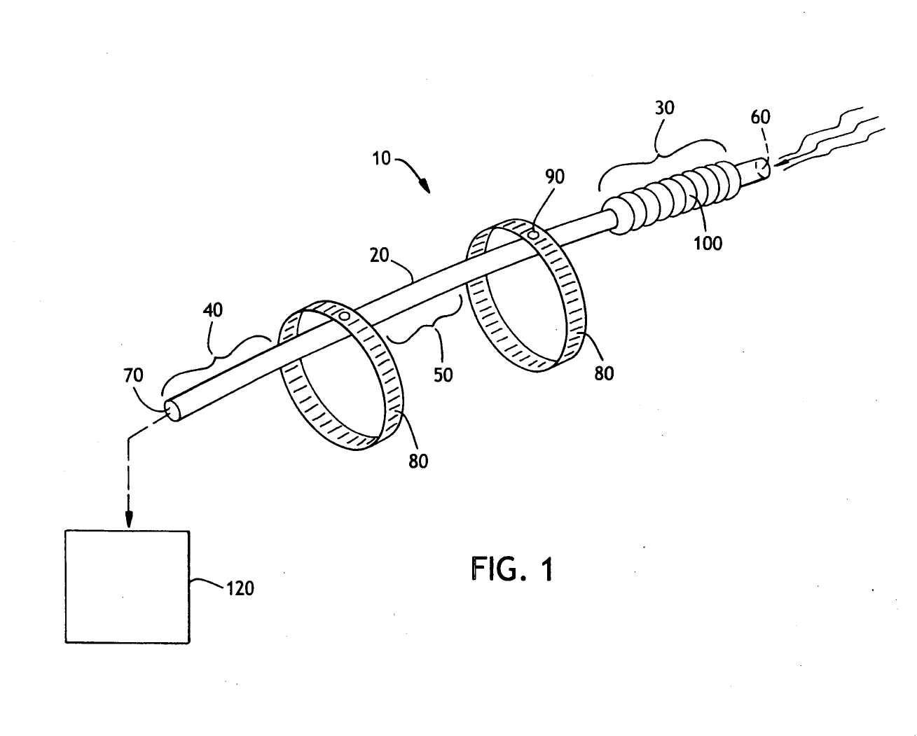

Referring to FIG. 1, a hands-free device for removing smoke from an operative site is provided. The apparatus 10 includes a tube having a tube body 20. The tube body 20 includes a distal end 30, a proximal end 40, and a substantially cylindrical central portion 50. Additionally, the tube has at least one distal opening 60 and at least one proximal opening 70.

The tube body may include any flexible material which is compatible with and does not damage the glove, gown, or skin over which the tube may come into contact with during use. These materials include, but are not limited to, latex, silicone, polyvinyl chloride, polyurethane, plastic, or polytetrafluoroethylene.

The tube may be manufactured by any method known in the art of manufacturing tubing. A non-limiting example of a manufacturing process suitable for this purpose is the coextrusion of two tubes (coextrusion being a process known and understood by those having skill in the manufacture of tubing), an outer tube and an inner tube. Additional, non-limiting examples include utilizing reinforcement inserts such as wire inside at least a portion of the shaft of the tube, utilizing a spiral wound reinforcement, or utilizing a stabilizing mesh incorporated into the wall of the tube.

Returning to FIG. 1, the apparatus also includes one or more holders 80 attached to the tube body. The holders engage with the covered or uncovered hand, wrist, forearm, or combinations thereof during use. The hand, wrist, forearm, or combinations thereof may be covered with a surgical gown and/or glove, for example. Non-limiting examples of holders include a rubber material, an elastic material, a non-elastic material, a cohesive material, a pressure sensitive adhesive, one or more mechanical fasteners, Velcro, or a hook and loop fastener.

The holder is attached to the tube body at one or more attachment zones 90. The holder may be attached to the attachment zone utilizing any number of mechanisms. Non-limiting examples include adhesives and hook and loop fasteners.

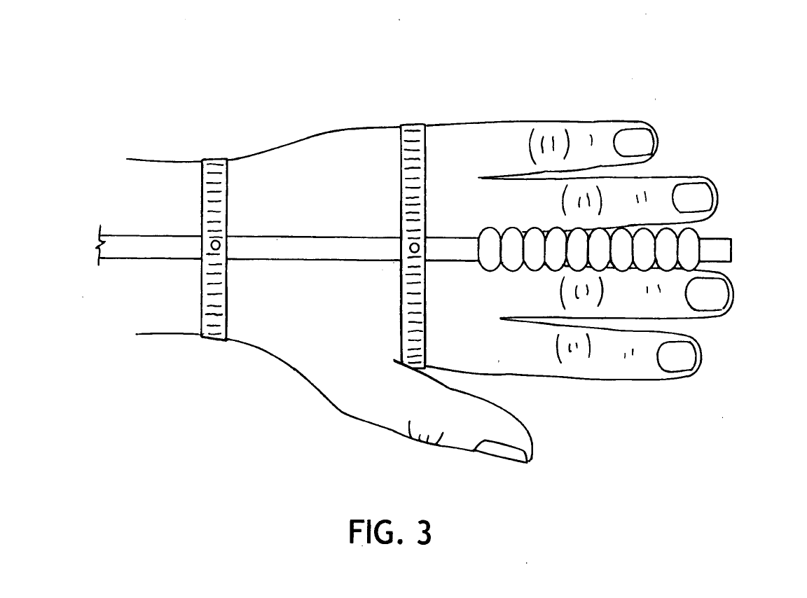

In use, the tube rests upon the dorsal portion of the hand (See FIG. 3), although it could rest upon any portion of the hand. The proximal opening 70 of the tube is attached to a suctioning device 120 such as for example, a smoke evacuator or wall suctioning unit, and the distal opening 60 of the tube is positioned to receive surgical smoke or other undesirable substances.

During use of the suctioning device, a vacuum is created by the suctioning device, and smoke, particulate matter associated with smoke, air, aerosols, chemical vapors, gaseous or generally gaseous material and/or non-liquid fluids, as well as other similar substances are drawn through the distal end, central portion, and proximal end of the tube into the suctioning device. Desirably, the device is not designed for fluid suctioning although some incidental fluid suctioning may occur during use. These substances may be generated by heating or burning of tissue, but may also be generated by released inert gas from endoscopic procedures, vaporized cooling liquids from laser procedures, or may be generated from other medical procedures. Once these substances are received into the suctioning device, the substances are filtered and clean air is released back into the ambient atmosphere, as known in the art.

Regardless of the type of tube utilized or the method of manufacturing the tube, it may be desirable for at least some portions of the tube to have different characteristics than other portions of the tube. For example, the distal end 30 and proximal end 40 of the tube may be made of material which exhibits a greater degree of rigidity than the substantially central portion, such as, for example, a stiffer formulation of plastic or polyurethane.

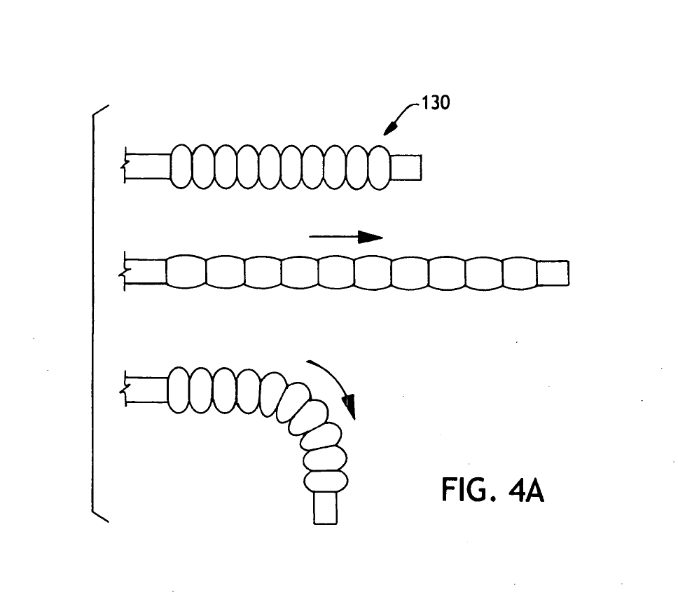

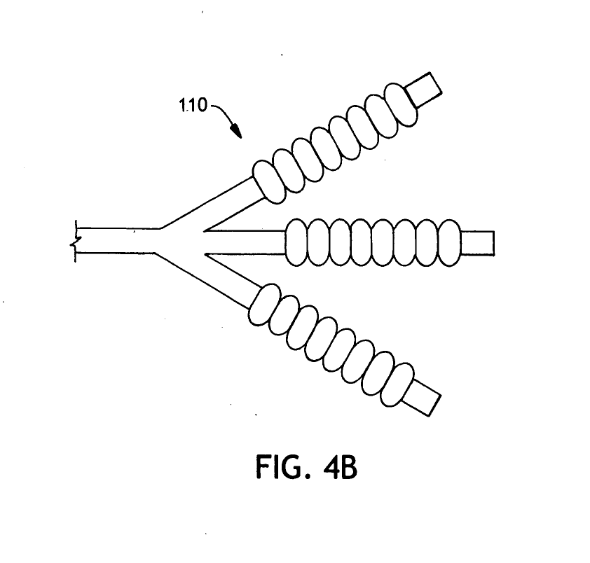

In an embodiment, the distal end of the apparatus may be composed of a corrugated material 100. As illustrated in FIG. 4A, corrugation 130 of the distal end allows the distal end to be extended, retracted, or bended in numerous ways, similar to, for example, a drinking straw. Advantageously, this allows the distal end of the tube to be manipulated as necessary to facilitate suctioning of surgical smoke while not interfering with the surgical procedure.

In an alternative embodiment, the distal opening of the apparatus may define a flexible screen. The screen is adapted to exclude the passage of surgical articles into the distal end of the tube. These surgical articles include surgical instruments, skin, surgical gown materials, sponges, and surgical drape materials. In this regard, the screen is desirably constructed so that only surgical smoke, particulate matter associated with surgical smoke, and air should be allowed to be suctioned into the distal end of the tube. The filter may be made from a variety of materials, including, but not limited to, for example, a flexible wire mesh.

In a further alternative embodiment, the distal opening may be constructed so that the tip of the distal opening may be closed when lateral force is exerted against a portion of the distal end that is proximal to the distal opening, similar to, for example, the closing of a milk carton. This will allow the suctioning mechanism to be shut off when it is not in use and further limit any chance of surgical materials beings accidentally suctioned into the tube.

Regardless of the construction of the distal opening, the size of the distal opening should be sufficient to suction surgical smoke. In this regard, the distal opening will desirably be about 0.25 to about 1.25 inches in diameter, but may be any size effective for suctioning surgical smoke. Additionally, surgical smoke is typically suctioned at a flow rate of about 2-50 cubic feet per minute (CFM), but may vary widely depending upon the degree and speed of suctioning desired. Additionally, the distal opening may be any shape effective for suctioning. These shapes include, but are not limited to round, square, or triangular.

Additionally, it may be desirable for the distal end to be in the shape of a Y-connector 110 (See FIG. 4B). A Y-connector 110 allows smoke, or other undesirable atmospheric substances to be advantageously suctioned into the tube by utilizing more than one opening. This allows for quicker, more efficient suctioning. Further, as an alternative to a Y-connector, the distal end my include 2 or more fingerlike projections. Additionally, a Y-connector may attached to the proximal end of the tube to accommodate the wearing of a smoke removal device on each arm, both being connected to a single suctioning unit.

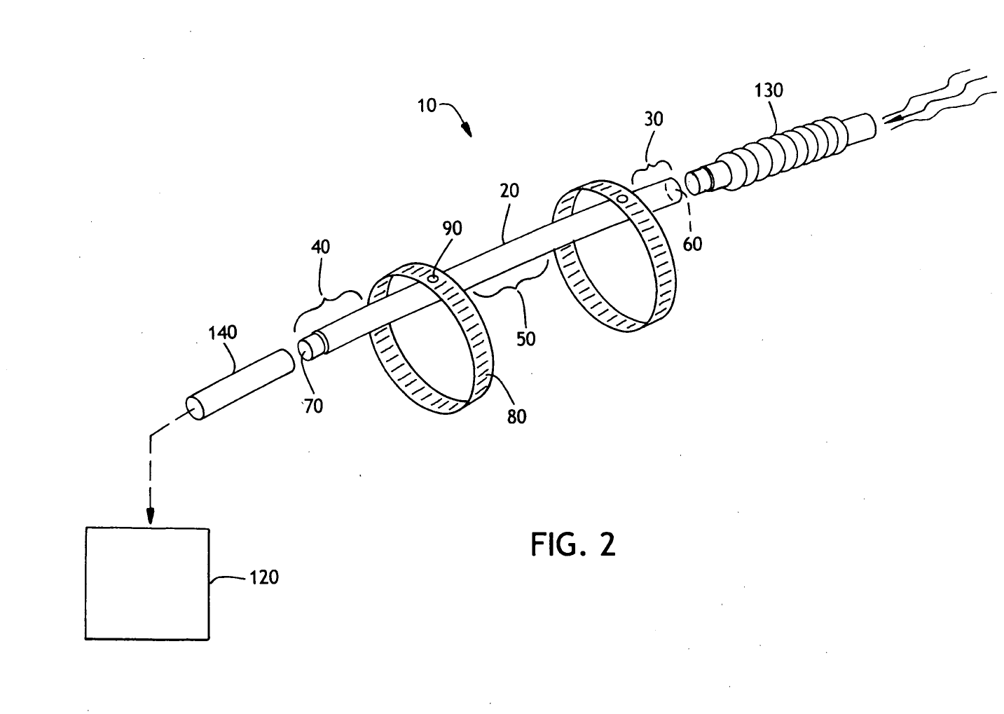

Turning now to FIG. 2, a hands-free device for removing smoke from an operative site is provided. The apparatus 10 includes a flexible tube having a tube body 20. The tube body 20 includes a distal end 30, a proximal end 40, and a substantially cylindrical central portion 50. Additionally, the tube has at least one distal opening 60 and at least one proximal opening 70.

The apparatus, desirably, may include at least one capture tube 130 and at least one evacuation tube 140 that are separate components from the flexible tube. In use, the capture tube is connected to the distal end of the flexible tube (by a male or female fit) in order to receive smoke therethrough. The capture tube may be made of a corrugated material and an opening within the tube may define a flexible screen. Additionally, the evacuation tube is connected to the proximal end of the flexible tube (by male or female fit) to evacuate smoke therethrough into a suctioning device adapted to capture and/or filter smoke.

Regardless of the form of the apparatus that is used, unibody construction or a flexible tube having separate capture and evacuation tubes, the apparatus may be packaged in a sterile container or may be sterilized prior to use, as known in the art. Additionally, during use, the apparatus remains in the sterile field and may be worn on one or both hands.

In another aspect of the invention, a method of using a hands-free apparatus for removing smoke from an operative site is provided for. The method includes providing a tube having a tube body comprising a distal end, a proximal end, a substantially cylindrical central portion, at least one distal opening, and a proximal opening. The method also includes attaching one or more holders to the tube body at one or more attachment zones so the tube body releasably engages with the hand, wrist, forearm, or combinations thereof during use. The method further includes receiving smoke through the distal opening of the tube and transmitting smoke through the proximal opening of the tube to a suctioning device.

What is claimed is:

1. A hands-free apparatus for removing smoke from an operative site, the apparatus comprising:
(a) a tube having a tube body comprising a distal end, a proximal end, a substantially cylindrical central portion, at least one distal opening, and a proximal opening;
(b) one or more holders attached to the tube body and adapted to releasably engage with the hand, wrist, forearm, or combinations thereof during use;
(c) one or more attachment zones whereon the holders are attached to the tube body,
wherein the distal opening of the tube is adapted to receive smoke therethrough and the proximal opening of the tube is adapted to be attached to a suctioning device.

2. The apparatus of claim 1, wherein the tube rests at least partially upon a dorsal portion of the hand, wrist, forearm, or combinations thereof during use.

3. The apparatus of claim 1, wherein the distal opening defines a screen adapted to exclude the passage of surgical articles into the distal end of the tube.

4. The apparatus of claim 1, wherein the distal end comprises a corrugated material.

5. The apparatus of claim 4, wherein the distal end and the proximal end of the apparatus exhibit a greater degree of rigidity than the substantially central portion.

6. The apparatus of claim 1, wherein at least one distal opening is adapted to be closed when lateral force is exerted against a portion of the distal end that is proximal to the distal opening.

7. The apparatus of claim 1, wherein the distal end is in the shape of a y-connector.

8. The apparatus of claim 1, wherein the holder comprises a rubber material, an elastic material, a non-elastic material, a cohesive material, a pressure sensitive adhesive, one or more mechanical fasteners, Velcro, or a hook and loop fastener.

9. The apparatus of claim 1, wherein the suctioning device is a smoke evacuator or wall suctioning unit.

10. A hands-free apparatus for removing smoke from an operative site, the apparatus comprising:
(a) a flexible tube having a tube body comprising a distal end and a proximal end, the distal end being adapted to connect to a capture tube for receiving smoke therethrough and the proximal end being adapted to connect to to a suctioning device adapted to capture received smoke;
(b) a capture tube adapted to connect to the distal end of the flexible tube for receiving smoke therethrough
(c) an evacuation tube adapted to connect to the proximal end of the flexible tube to evacuate smoke therethrough to a suctioning device adapted to capture smoke
(d) one or more holders attached to the tube body and adapted to releasably engage with the hand, wrist, forearm, or combinations thereof during use;
(e) one or more attachment zones whereon the holders are attached to the tube body.

11. The apparatus of claim 10, further comprising a distal opening adapted to enable a male or female fit of the distal end of the flexible tube into the capture tube.

12. The apparatus of claim 11, further comprising a proximal opening adapted to enable a male or female fit of the proximal end of the flexible tube into the evacuation tube.

13. The apparatus of claim 10, wherein the flexible tube rests at least partially upon a dorsal portion of the hand, wrist, forearm, or combinations thereof during use.

14. The apparatus of claim 11, wherein the capture tube further comprises a screened opening adapted to exclude the passage of surgical articles into the distal end of the flexible tube.

15. The apparatus of claim 10, wherein the capture tube comprises a corrugated material.

16. The apparatus of claim 10, wherein the capture tube and the evacuation tube exhibit a greater degree of rigidity than the flexible tube.

17. The apparatus of claim 10, wherein the capture tube is in the shape of a y-connector.

18. The apparatus of claim 10, wherein the holder comprises a rubber material, an elastic material, a non-elastic material, a cohesive material, a pressure sensitive adhesive, one or more mechanical fasteners, Velcro, or a hook and loop fastener.

19. The apparatus of claim 10, wherein the suctioning device is a smoke evacuator or wall suctioning unit.

20. A method of using a hands-free apparatus for removing smoke from an operative site, the method comprising:
(a) providing a tube having a tube body comprising a distal end, a proximal end, a substantially cylindrical central portion, at least one distal opening, and a proximal opening;
(b) attaching one or more holders to the tube body at one or more attachment zones so the tube body releasably engages with the hand, wrist, forearm, or combinations thereof during use;
(c) receiving smoke through the distal opening of the tube
(d) transmitting smoke through the proximal opening of the tube to a suctioning device.
